(19) United States
(12) Patent Application Publication
(10) Pub. No.: US 2012/0316382 A1
(43) Pub. Date: Dec. 13, 2012

(54) Footwear with Magnets Embedded

(76) Inventor: Raphael Edwards, New Rochelle, NY (US)

(21) Appl. No.: 13/155,989

(22) Filed: Jun. 8, 2011

(51) Int. Cl. A61N 2/00

(52) U.S. Cl. 600/15

(57) ABSTRACT

A device and method for positioning or embedding magnets in sneakers, shoes, sandals and other outer footwear is disclosed and claimed.

### BACKGROUND

Magnet therapy or magnotherapy is an alternative medical therapy in which the placement of magnets or magnetic devices on the skin is thought to prevent or treat symptoms of disease, especially pain. Various configurations and devices exist for positioning magnets near particular portion(s) of the body. For example U.S. Pat. No. 6,258,020 discloses many configurations for positioning magnets on various articles of clothing and bands. For positioning magnets near a foot it discloses magnets embedded in “sock-like-footwear” and an “ankle-to-foot support” band. Repeated washing and drying of “sock-like-footwear” presents a problem. And the use of a band to strap on requires additional action when dressing and additional items to lose or forget.

### BRIEF DESCRIPTION OF DRAWINGS

FIG. 1 depicts a sneaker with magnets embedded.

FIG. 2 depicts a shoe with magnets embedded.

FIG. 3 depicts a boot with magnets embedded.

### DESCRIPTION OF INVENTION

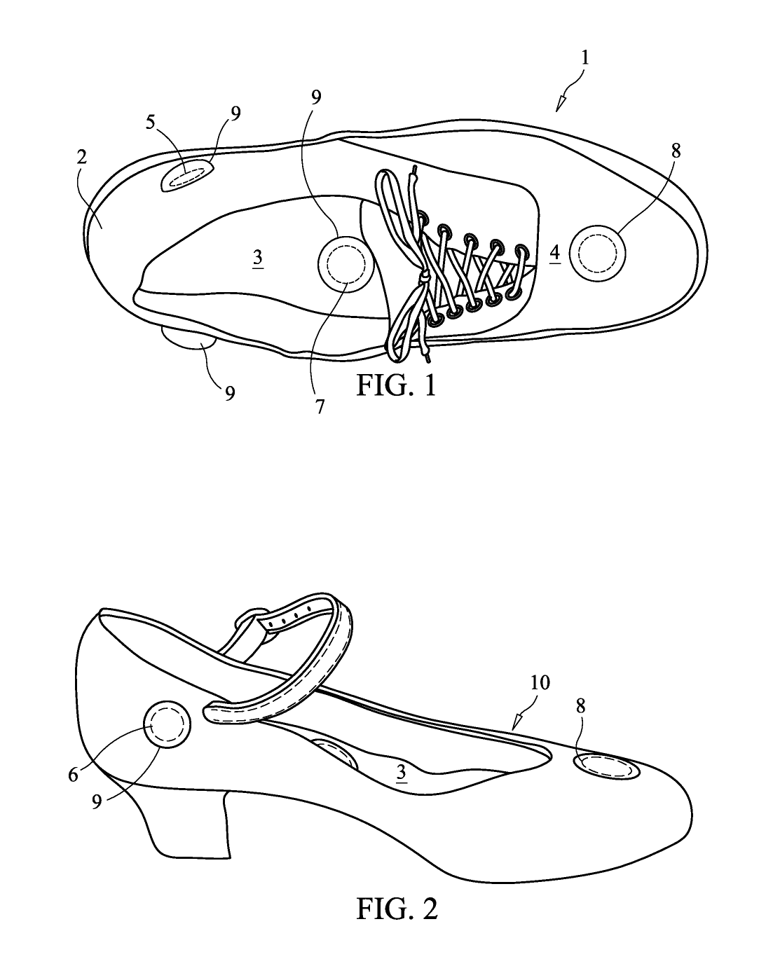

In the embodiment shown in FIG. 1, magnets (5-8) are embedded into a sneaker (1). Two magnets (5 and 6) are embedded on the side of the sneaker (1) in the heel area (2). A third magnet (7) is embedded in the sole (3) of the sneaker (1). A fourth magnet (8) is embedded on the top (4) of the sneaker (1) which is typically a tongue.

The number and location of the magnets can be changed as warranted by the area(s) to be targeted and size of the foot. Each magnet is typically placed in a pod (9) which is an appropriately sized container embedded in the sneaker. Or the magnets can be directly embedded into voids created in the sneaker.

The magnets can be embedded in any outer footwear including shoes, slippers, sandals, boots, etc. The outer footwear in the embodiment in FIG. 2 is a shoe (10).

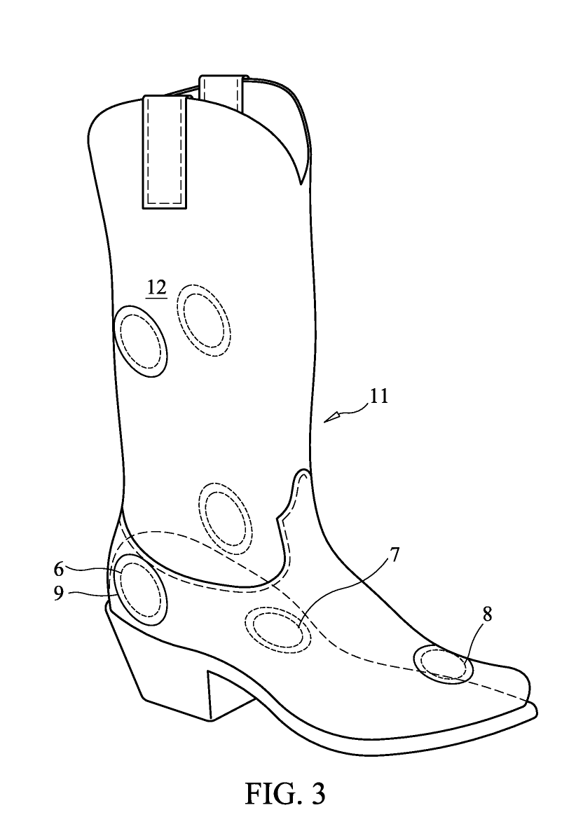

For the embodiment in FIG. 3 magnets are embedded in a boot (11). Depending on the height of the boot, additional magnets can be added to apply magnet therapy to other areas of the body such as the ankle and calf (12). The above illustrates various concepts, structures and techniques which are the subject of this patent. It will now become apparent to those of ordinary skill in the art that other embodiments incorporating these concepts, structures and techniques may be used. Accordingly, it is submitted that that scope of the patent should not be limited to the described embodiments but rather should be limited only by the spirit and scope of the following claims.

I claim:

1. A magnetic therapy enclosure for the foot comprising:
an outer footwear having a heel area, a sole and a top;
a minimum of 1 pod embedded in the footwear; and
a magnet inserted in each pod.

2. A magnetic therapy enclosure according to claim 1 wherein at least one magnet is located on each side of the outer footwear in the heel area.

3. A magnetic therapy enclosure according to claim 2 wherein at least one magnet is located in the sole.

4. A magnetic therapy enclosure according to claim 3 wherein at least one magnet is located in the top.

5. A magnetic therapy enclosure according to claim 4 wherein at least one magnet is located on each of side on the footwear in the heel area.

6. A magnetic therapy enclosure according to claim 5 in which the outer footwear is a sneaker.

7. A magnetic therapy enclosure according to claim 5 in which the outer footwear is a shoe.

8. A magnetic therapy enclosure according to claim 5 in which the outer footwear is a slipper.

9. A magnetic therapy enclosure according to claim 5 in which the outer footwear is a sandal.

10. A magnetic therapy enclosure according to claim 5 in which the outer footwear is a boot.

11. A magnetic therapy enclosure according to claim 10 wherein the outer footwear includes an ankle area and a calf area.

12. A magnetic therapy enclosure according to claim 11 in which at least one magnet is located in the ankle area.

13. A magnetic therapy enclosure according to claim 11 in which at least one magnet is located in the calf area.

14. A method for applying magnetic therapy to areas of the body surrounded by footwear comprising:
selecting the footwear including a top area, a heel area and a sole area;
placing a pod in at least one area of the footwear, and
placing a magnet in each pod.

15. A method for applying magnetic therapy according to claim 14, wherein at least one pod with magnet is located the top area, the heel area and the sole area of the footwear.

16. A method for applying magnetic therapy according to claim 15, wherein the footwear is a boot which includes a calf area with at least one magnet in the calf area.

17. A method for applying magnetic therapy according to claim 14, wherein the footwear includes an ankle area with at least one magnet in the ankle area.

18. A magnetic therapy enclosure for the foot comprising:
an outer footwear having a heel area, a sole and a top;
a minimum of 1 magnet embedded in the footwear.

19. A magnetic therapy enclosure according to claim 18, wherein at least one magnet is located in the top, at least one magnet is located in the heel area, and at least one magnet is located in the sole of the footwear.

20. A magnetic therapy enclosure according to claim 19 the footwear is a boot which includes a calf area and an ankle area with at least one magnet in each of these areas.
